(19) United States
(12) Patent Application Publication
(10) Pub. No.: US 2010/0272689 A1
(43) Pub. Date: Oct. 28, 2010

(54) Protease Having Algicidal Activity, Gene Encoding the Same and Algicidal Formulation Comprising the Same

(75) Inventors: Sang-Jin Kim, Seoul (KR); Sung-Gyun Kang, Gyeonggi-do (KR); Kae-Kyoung Kwon, Gyeonggi-do (KR); Jung-Hyun Lee, Gyeonggi-do (KR); Hyun-Sook Lee, Gyeonggi-do (KR); Ji-Hyun Kang, Gyeonggi-do (KR); Jae-Hak Sohn, Busan (KR)

Correspondence Address: CLARK & ELBING LLP, 101 FEDERAL STREET, BOSTON, MA, 02110, US

(73) Assignee: KOREA OCEAN RESEARCH & DEVELOPMENT INSTITUTE, Ansan-si, Gyeonggi-do (KR)

(21) Appl. No.: 12/664,194

(22) PCT Filed: Apr. 25, 2008
(86) PCT No.: PCT/KR2008/002370

§ 371 Date: Feb. 19, 2010

(30) Foreign Application Priority Data
Jun. 11, 2007 (KR) 10-2007-0056918

(51) Int. Cl. A01N 63/00; C12N 9/48; C12N 15/57; A01P 13/00

(52) U.S. Cl. 424/93.4; 435/212; 536/23.2; 424/94.63

(57) ABSTRACT

The present invention relates to a protease having algicidal activity, a gene encoding the same and algicidal formulation comprising the same. The protease according to the present invention showed high algicidal activity. Therefore, the protease can be used treatment of red tide in marine region of red tide occurrence.

### TECHNICAL FIELD

The present invention relates to a protease inhibiting the growth of red tide organisms and a gene encoding the same.

### BACKGROUND ART

The species that cause marine red tide is belonging to Bacillariophyceae, Raphidophyceae and Dinophyceae, especially Flagellates which is most of these species. Research on algicidal bacteria using the soft-agar overlay method has been limited because Bacillariophyceae can grow on a medium containing agar, but Flagellates can't grow.

Algicidal bacteria isolated from marine and coastal environments have been assigned to the genera Alteromonas, Bacillus, Caulobacter, Cytophaga, Flavobacterium, Pseudoalteromonas, Pseudomonas, Saporospira and Vibrio. Algicidal bacteria isolated from marine are divided phylogenetically into two groups, Flavobacterium-Cytophaga complex and Proteobacteria branch (e.g., Alteromonas, Pseudomonas, Pseudoalteromonas, Saporospira and Vibrio).

Generally, Flavobacterium-Cytophaga complex having degradation activity of various biomacromolecules are considered to be closely related to plant plankton because they can use algal cell wall and exudates discharged from itself as a nutrient source.

According to recent research, the algicidal mechanism of algicidal bacteria can be mainly divided into the following two categories: (1) a mechanism by contact in which algicidal bacteria adhere to the surface of red tide organisms to lyse the red tide organisms (e.g., Cytophaga and Alteromonas), and (2) a mechanism in which algicidal bacteria secrete algicidal substances extracellularly to induce the growth inhibition or lysis of red tide organisms. Most of algicidal bacteria are included in the second mechanism. Most of algicidal bacteria isolated from marine have pathways to product extracellular materials, but there has been no report of isolation, identification and production pathway of these materials.

The present inventors isolated Kordia algicida OT-1, algicadal bacteria that can kill Skeletonema costatum, and deposited on Apr. 24, 2008, by the Korean Collection for Type Cultures (KCTC) as KCTC Deposit No. KCTC 11320BP.

Sequencing analysis based on 16S-rRNA and similarity search to stains registered in NCBI (national center for biotechnology information) showed that Kordia algicida OT-1 was a novel strain in the family Flavobacteria [Sohn et al., Int. J. Syst. Evol. Microbiol., 54:675-680, 2004].

### DISCLOSURE

### Technical Problem

It is an object of the present invention to provide a novel protein inhibiting the growth of red tide organisms and genes encoding the protein.

It is another object of the present invention to provide an algicidal formulation comprising the protein as an active ingredient.

It is another object of the present invention to provide a method for treatment of red tide using the said algicidal formulation.

### Technical Solution

The present invention provides a protease having algicidal activity, comprising any one amino acids sequence selected from the group consisting of SEQ ID NO: 1, SEQ ID NO: 4 to SEQ ID NO: 7, SEQ ID NO: 26 and SEQ ID NO: 27, and a gene encoding the same.

Also, the present invention provides algicidal formulation comprising said protease as an active composition.

Also, the present invention provides a method for controlling red tide comprising a step of introducing the algicidal formulation to the marine region where occurred red tide. More specifically, the present invention provides a method for controlling red tide comprising the steps of: (a) introducing one or more proteases selected from the group consisting of SEQ ID NO: 1, SEQ ID NO: 7 and SEQ ID NO: 27 using Kordia algicida OT-1 and (b) introducing an effective amount of algicidal formulation comprising said protease produced by above step. Said algicidal formulation can be introduced into marine region where red tide occurred with Kordia algicida OT1 KCTC 11320BP simultaneously or with an effective amount of protease purely or partially isolated from culture product of Kordia algicida OT1 KCTC 11320BP. To meet conditions similar to seawater, the said algicidal formulation could contain 0.5˜6% NaCl, 0.05 g-1.2 g/1 Mg²⁺ and 0.05-1.0 g/l Ca²⁺.

### DESCRIPTION OF DRAWINGS

FIG. 1 shows the result of native PAGE (12%) analysis for isolating proteins of Kordia algicida OT1 KCTC 11320BP, an algicidal bacteria.

FIG. 2 shows the result of SDS-PAGE analysis to the proteins of band No. 3 and No. 10 of FIG. 1, having algicidal activity, under denaturing condition.

FIG. 3 shows the amino acid sequence of SEQ ID NO: 1, named KAOT1_{—}10476.

FIG. 4 shows schematic pictures of several gene constructs for identifying some characteristics of KAOT1_{—}10476 protein.

FIG. 5 shows the result of SDS-PAGE analysis to the proteins encoded by 10476_{—}1 to 10476_{—}9.

FIG. 6 shows the result of refolding of proteins encoded by 10476_{—}1 and 10476_{—}6 to 10476_{—}9 to solubilize them.

FIG. 7 shows protease activity of said proteins (10476_{—}1 and 10476_{—}6 to 10476_{—}9).

FIG. 8 shows the processing of 10476_{—}8 protein for a week.

FIG. 9 and FIG. 10 show protease activity of 10476_{—}7 protein under various temperature and pH conditions.

FIG. 11 and FIG. 12 show protease activity of 10476_{—}7 protein under various concentrations of metal ions (zinc or calcium ion).

FIG. 13 and FIG. 14 show the homology among KAOT1_{—}10476 protein (Kordia algicida OT1) and proteins from various marine organisms such as Flavobacterium johnsoniae UW101, Croceibacter atlanticus HTCC2559, and Leeuwenhoekiella blandensis MED217.

FIG. 15 shows the result of PCR amplification to express proteins of marine organisms having algicidal activity and homology with KAOT1_{—}10476 protein.

FIG. 16 shows the expression of proteins of marine organisms having algicidal activity and homology with KAOT1_{—}10476 protein.

### BEST MODE

The present invention provides a protease having algicidal activity and amino acids sequence of SEQ ID NO: 1, isolated from Kordia algicida OT1 KCTC 11320BP.

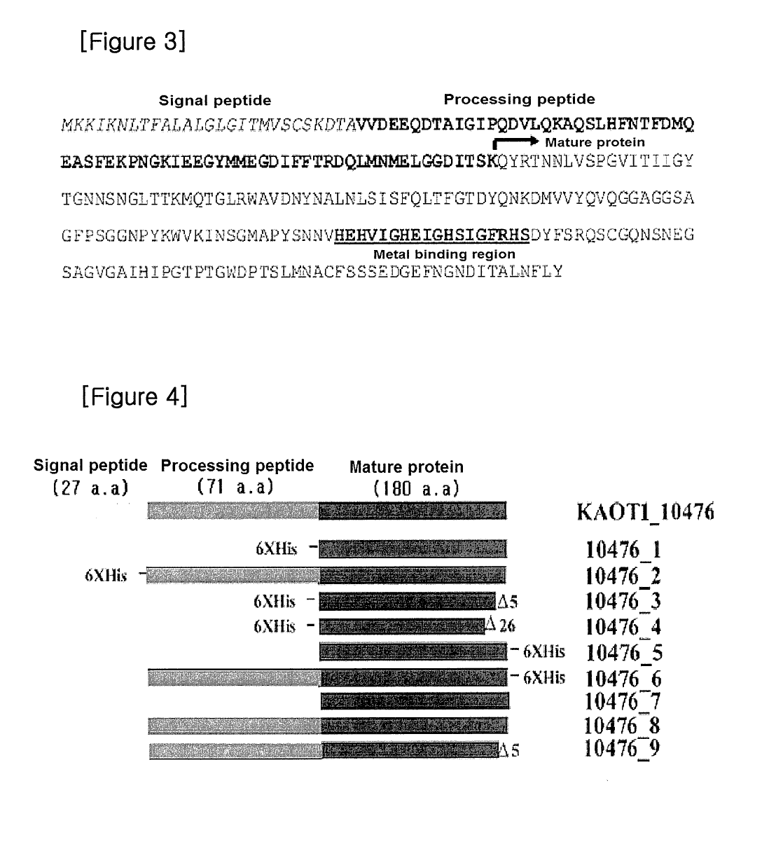

Amino acid residues 1-27 of SEQ ID NO: 1 respond to signal peptide part, amino acid residues 28-98 responding to processing peptide part and amino acid residues 99-278 responding to mature protein part. Said mature protein includes metal binding region (amino acid residues 200-217) (FIG. 3).

The protease according to the present invention may have deletions, substitutions, or additions of amino acid residues without affecting the activity of protease, and could be used a partial fragment of the protein in certain purpose. Such modified proteases also are in the scope of present invention. Therefore, the protease according to present invention includes all polypeptides having substantially same amino acids sequence or a partial fragment of said protease protein.

Therefore, the protease according to present invention includes the algicidal protease of amino acids sequence of SEQ ID NO: 26 which does not have the signal peptide part. Also, the protease according to present invention includes the protease having amino acids sequence of SEQ ID NO: 27 which doesn't have the parts of signal peptide and processing peptide.

The genes encoding the protease could be variously modified in codon region in consideration of codon degeneracy or preferred codon usage in a organism to which the protease is intended to be expressed, provided that the amino acids sequence of the protein is not altered or could be modified or altered in a region except coding region, provided that it does not affect the expression of the protease. All of these modified genes are in the scope of present invention. Therefore, the present invention provides substantially same polynucleotide sequences encoding amino acids sequence of SEQ ID NO: 1, and the fragments thereof. More preferably, the present invention provides polynucleotides having nucleotide sequence of SEQ ID NO: 2.

Also, the present invention provides polynucleotides having nucleotide sequences of SEQ ID NO: 28 and 29 which encode the algicidal proteases having amino acids sequence of SEQ ID NO: 26 and 27.

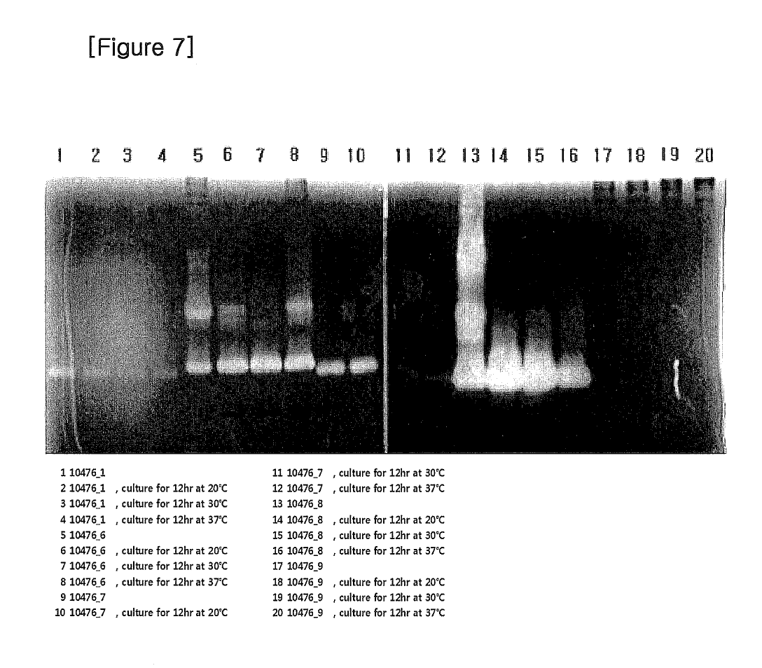

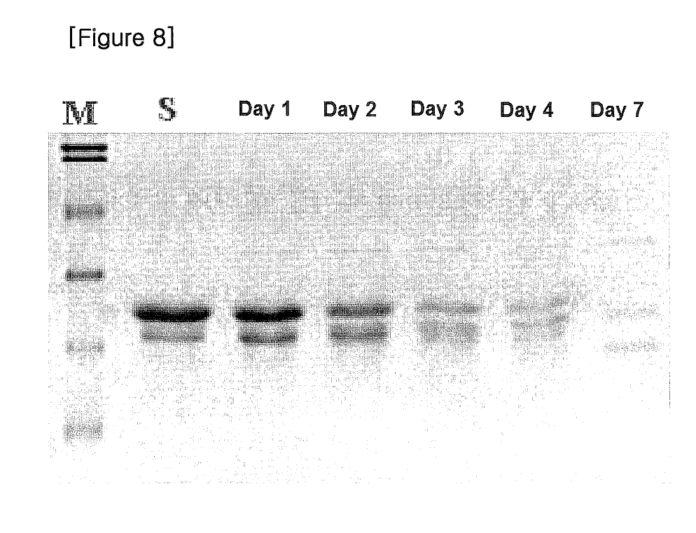

The protease of SEQ ID NO: 1 shows higher proteolytic activity in the case that it contains a processing peptide part in addition to a mature protein part. The protein including processing peptide and mature protein changes to mature protein by autocleavage with the lapse of time (FIGS. 7 and 8). It is found that the C-terminal of mature protein play an important role in protein processing.

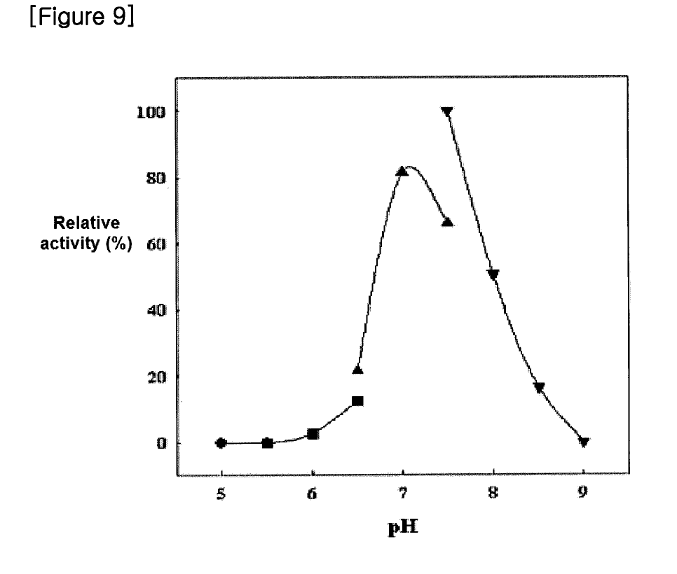

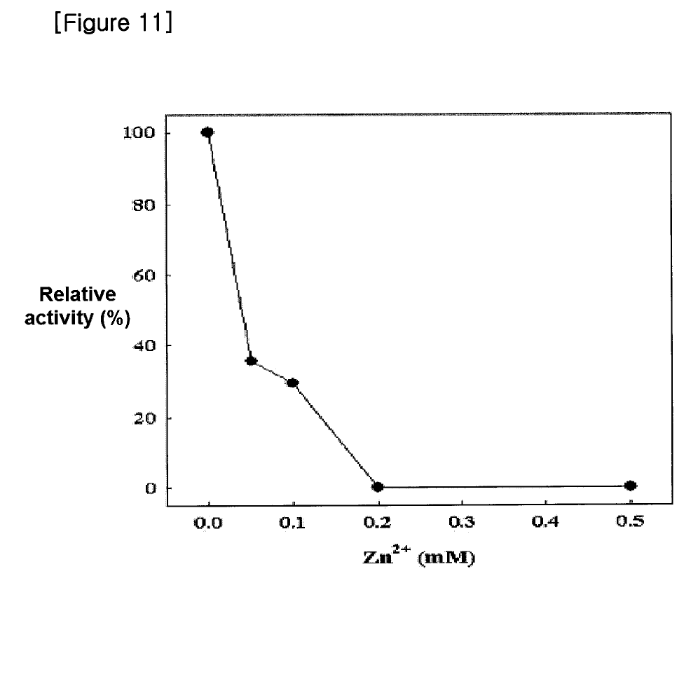

Also, the protease according to the present invention exhibits optimum activity in the pH range of 6.5-8.0, preferably in the pH range of 7.0-8.0, in the temperature range of 18-28° C., preferably in the temperature range of 20-23° C., most preferably in the temperature range of 20° C., and in the presence of 80-100 mM calcium ion (FIGS. 9, 10 and 12). It is found that zinc ion inhibits the activity of the protease (FIG. 11).

The protease having amino acids sequence of SEQ ID NO: 1 according to the present invention shows efficient algicidal activity against algae species including Cochlodinium polykrikoides, Thalassiosira sp., Heterosigma akashiwo, Skeletonema costatum, Alexandrium sp., Chaetoceros cuvisetus and Gymnodium sp, etc.

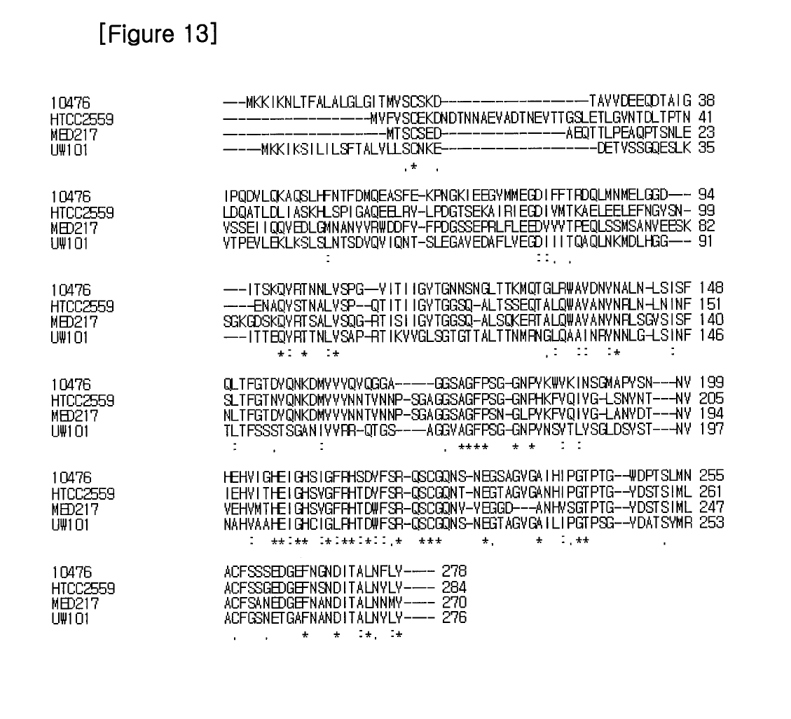

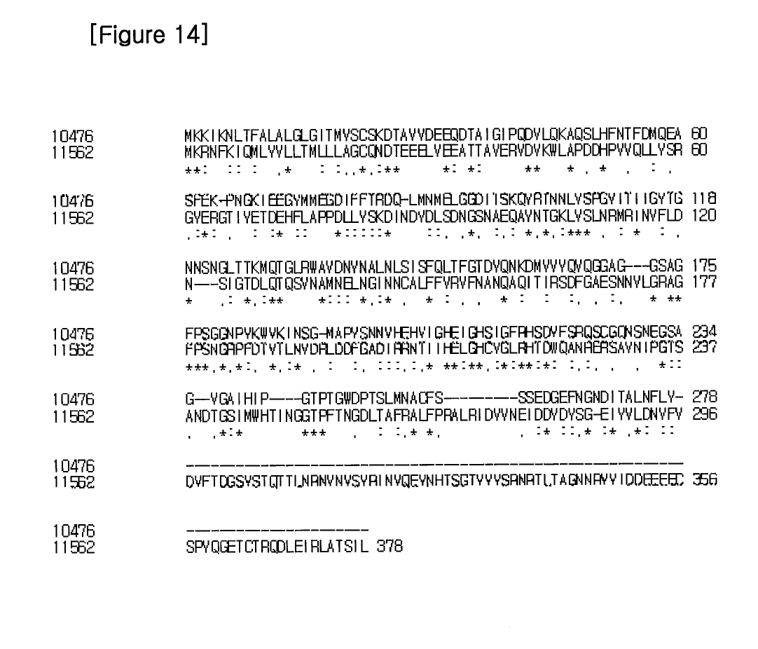

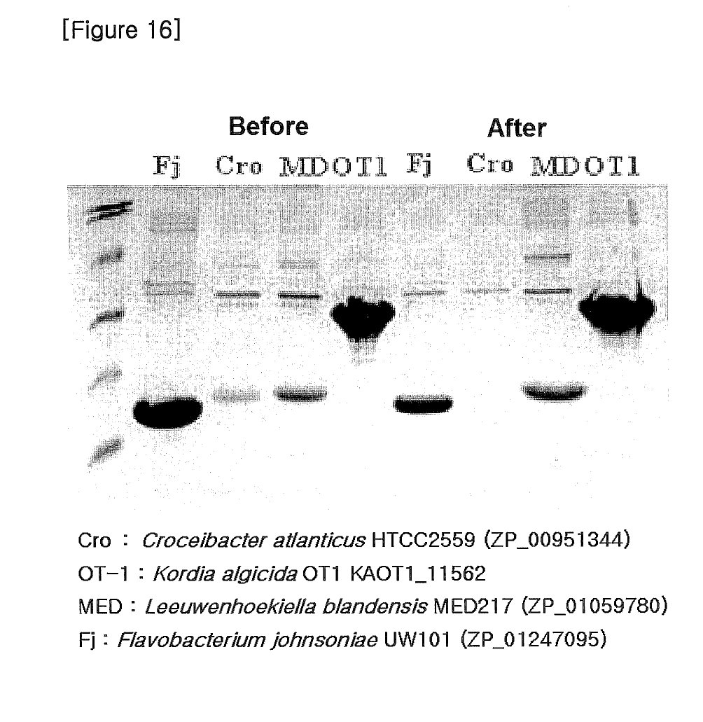

The protease having amino acids sequence of SEQ ID NO: 1 exhibits more than 40% identity with proteins of Flavobacterium johnsoniae UW101 (ZP_{—}01247095, SEQ ID NO: 4), Croceibacter atlanticus HTCC2559 (ZP_{—}00951344, SEQ ID NO: 5) and Leeuwenhoekiella blandensis MED217 (ZP_{—}01059780, SEQ ID NO: 6) in more than 80% gene coverage. Especially, after peptide processing the mature protease shows more than 50% of homology with said proteins (FIG. 13). Also, the protease shows high homology to Kordia algicida KAOT1_{—}11562 (SEQ ID NO: 7) (FIG. 14). Also, expression patterns and activities of said proteins are highly similar to the pattern of the protein having amino acids sequence of SEQ ID NO: 1 (FIG. 16).

As described above, genes encoding each proteins could be modified and altered in a condition that the sequences of said proteins were not changed, the present invention provides substantially same polynucleotide sequences encoding amino acids sequence of SEQ ID NO: 4-7, and the fragments. More preferably, the present invention provides polynucleotides having nucleotide sequence of SEQ ID NO: 8-11.

Since proteases according to the present invention shows high algicidal activity, they and the genes encoding them are useful for control of red tide.

Also, the present invention provides algicidal formulation comprising the protein having one of amino acids sequence selected from the group consisting of SEQ ID NO: 1 and SEQ ID NO: 4 to SEQ ID NO: 7 as a active composition. The active composition can be included in a range of 1-99 wt % based on the total weight of formulation.

Also, the present invention provides a method for controlling red tide comprising the steps of introducing the algicidal formulation to the marine region where red tide occurred.

In the method according to the present invention, the used amount of said algicidal formulation could be varied according to the accumulation rates of organic matter or the occurrence rate of red tide, etc.

The said algicidal composition could be introduced in a various way such as; scattering at sea level, or introducing the bottom of the sea by using pipes, but is not limited thereto. Depending upon the type of algicidal formulation, and conditions of sea water or a lake, the method could be applied in a various way.

Hereinafter, the present invention will be described in further detail with reference to examples. It is to be understood, however, that these examples are for illustrative purposes only and are not to be construed to limit the scope of the present invention.

### MODE FOR INVENTION

### Example 1

### Culture of Algicidal Bacteria

Kordia algicida OT-1 KCTC 11320BP was cultured in ZoBell 2216 e agar medium [Oppenheimer, C. H., et al., J. Mar. Res., 11:10-18, 1952].

To prepare the preculture, 3% (v/v) of the culture was inoculated into 100 ml ZoBell 2216 e broth and incubated for 24 h in a shaking incubator (25° C., 150 rpm). 3% (v/v) of the preculture was added to 5 l jar fermenter (Korea Fermenter Inc., Korea) containing 3 l of ZoBell 2216 e broth, and then incubated at 25° C., 300 rpm and 0.5 vvm [Oppenheimer, C. H., et al., J. Mar. Res., 11:10-18, 1952].

### Example 2

### Preparation of Red Tide Organisms

Organisms used as host for algicidal bacteria screen are Cochlodinium polykrikoides, Thalassiosira sp., Heterosigma akashiwo, Skeletonema costatum, Alexandrium sp., Gymnodium sp., Chaetoceros cuvisetus. Each algae cell was grown to log phase in F/2 broth (0.075 g NaNO₃, 0.005 g NaH₂PO₄?H₂O, 0.030 g Na₂SiO₃?9H₂O, 1 ml F/2 trace metal solution, 1 ml F/2 vitamin solution and 1,000 ml aged seawater) [Stein, Handbook of phycological methods, Cambridge Univ. Press, 1973].

The cultured red tide organisms were diluted in F/2 broth to a final density of 0.05 (excitation wavelength=434 nm, emission wavelength=670 nm)on the fluorescent intensity using fluoro-spectrophotometer (Hitachi, Japan), and then 2.7 ml aliquots were dispensed into several test tubes (110 mm×10 mm) to prepare for the MPN (most probable number) method.

### Example 3

### Selection of Proteins having Algicidal Activity

3-1 Purification and Isolation of Proteins from Algicidal Bacteria

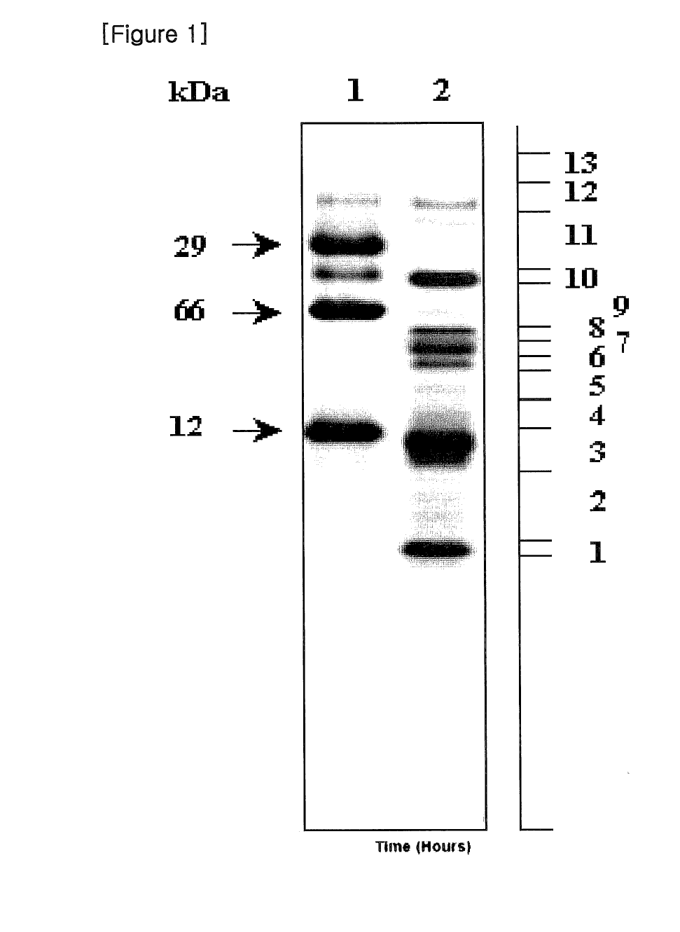

To isolate the protein having algicidal activity from algicidal bacteria cultured according to Example 1, said algicidal bacteria was cultured for 30 hr using 5 l jar fermenter. The component greater than 10 kDa in molecular weight was concentrated using ultrafiltration kit, and then proteins were seperated using native PAGE (12%). The results are shown in FIG. 1 (Lane 1, molecular size marker; Lane 2, proteins purified from algicidal bacteria).

### 3-2 Measurement of Algicidal Activity of Purified Proteins

With purified proteins as described above, 10% native polyacrylamide gel electrophoresis was carried out at a constant current of 20 mA for 100 min, and then some of gels were stained with Coomassie brilliant blue R-250 to check the position of the band. The other gels were cut into 13 small pieces according to band size, and then 1.5 ml of 20 mM Tris-HCl buffer (pH 8.0) was added to each sample, followed by stand for 2 hr at 4° C. in a refrigerator. The samples were centrifuged, and gels were precipitated. Then, the supernatant was concentrated using Ultracon (cut-off size 10 kDa). Algicidal activity of the concentrated samples was measured by the lawn assay with Skeletonema costatum. The culture of algicidal bacteria was used as a positive control, and ZoBell 2216 e broth, F/2 broth or 20 mM Tris-HCl (pH 8.0) buffer were used as a negative control according to condition of samples.

Specifically, Skeletonema costatum was cultured for 1-2 weeks at 20° C. under a 14 L (5000 lux)/10 D photoperiod. Each algicidal activity was compared by measuring the diameter of the clear zone on the lawn of Skeletonema costatum. The results are shown in Table 1. The size of clear zone (diameter, mm): −(no clear zone); +(1m); ++(2 mm); +++(3 mm); ++++(5 mm).

TABLE 1

| Band number | Algicidal |
|---|---|
| in FIG. 1 | activity |
| 13 | − |
| 12 | − |
| 11 | + |
| 10 | +++ |
| 9 | + |
| 8 | − |
| 7 | − |
| 6 | − |
| 5 | − |
| 4 | − |
| 3 | ++ |
| 2 | + |
| 1 | − |
| Negative control | − |
| Positive control | ++++ |

As shown in Table 1, proteins of band number 3 and 10 showed high algicidal activity.

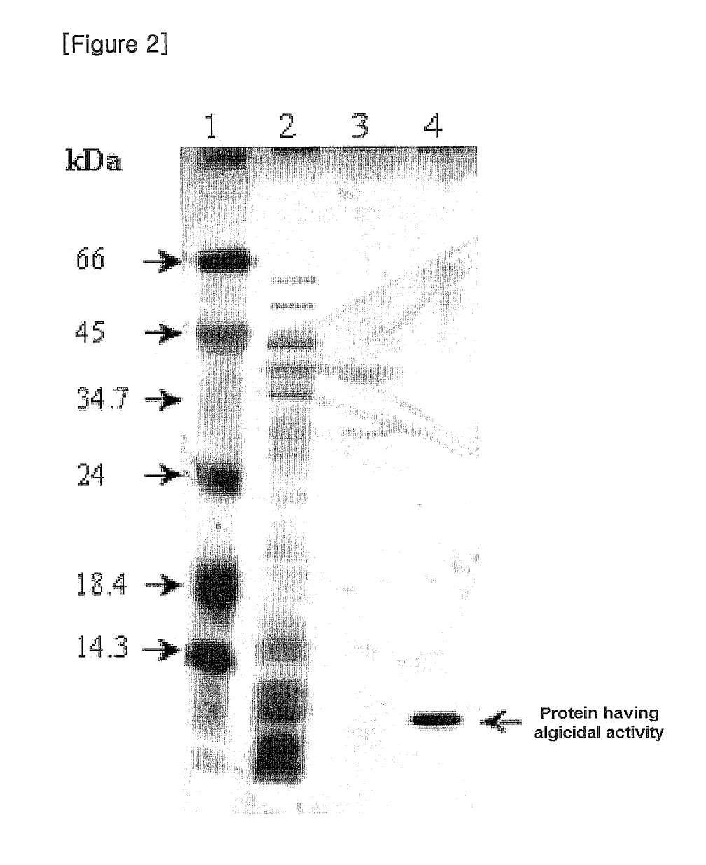

Also, SDS-PAGE was performed with proteins of band number 3 and 10 under denaturing conditions [Laemmli, Nature, 227:680-685, 1970] and expression of the protein of band number 10 was confirmed. The results are shown in FIG. 2 (Lane 1, size marker; Lane 2, proteins from algicidal bacteria; Lane 3, the protein of band number 3; Lane 4, the protein of band number 10). SEQ ID NO: 3 containing 20 amino acids was obtained by N-terminal amino acid sequencing analysis.

Said amino acid sequence revealed no significant homology with other proteins in a homology search of protein sequence databases such as NCBI Blast, PDF and SWISSPROT.

### Example 4

### Identification of Open Reading frame (ORF) Encoding the Protein having Algicidal Activity

Analysis of whole-genome sequencing of Kordia algicida OT1 (KCTC 11320BP) and data mining using N-terminal amino acid sequence of SEQ ID NO: 3 were performed. As a result, 18 of 20 amino acids of SEQ ID NO: 3 are identical with KAOT1_{—}10476protein (SEQ ID NO: 1).

### Example 5

### Construction of an Expression Vector

As shown in FIG. 3, KAOT1_{—}10476 protein of SEQ ID NO: 1 was expected to be a metallo protease consisting of signal peptide (amino acid residues 1-27), processing peptide (amino acid residues 28-98) and mature region (amino acid residues 99-278). Also, N-terminal sequence isolated from wild type was expected to be a mature protease that processing peptide was removed by extracellular secretion.

Therefore, to check algicidal activity of said protein, each expression vector was constructed as described below for expression of various proteins as shown in FIG. 4.

Firstly, various DNAs encoding KAOT1_{—}10476 protein of SEQ ID NO: 1 or its fragments were amplified by PCR using primers designed to have NdeI and XhoI restriction sites as shown in Table 2. The gene of SEQ ID NO: 1 was used as a template and TLA1 polymerase (Bioneer Inc., Korea) was used. The condition of reaction was 2 min at 95° C., 1 min at 55° C. and 1 min at 72° C. and 30 cycles of reaction were performed.

TABLE 2

| PCR product | Primer | SEQ ID No. |
|---|---|---|
| 10476_1 | OT1m_t | 12 |
| | OT1m_bc | 13 |
| 10476_2 | OT1p_t | 17 |
| | OT1m_bc | 13 |
| 10476_3 | OT1m_t | 12 |
| | OT1delta5_bc | 15 |
| 10476_4 | OT1m_t | 12 |
| | OT1delta26_bc | 16 |
| 10476_5 | OT1m_t | 12 |
| | OT1m_bH | 14 |
| 10476_6 | OT1p_t | 17 |
| | OT1m_bH | 14 |
| 10476_7 | OT1m_t | 12 |
| | OT1m_bc | 13 |
| 10476_8 | OT1p_t | 17 |
| | OT1m_bc | 13 |
| 10476_9 | OT1p_t | 17 |
| | OT1delta5_bc | 15 |

Secondly, pET24a expression vector was digested with NDeI and SalI and purified using plasmid purification kit (Quagen, Inc.), and then ligated them with each PCR product obtained above, and transformed into E. coli DH5α. DNAs extracted from the above transformants were purified using plasmid purification kit, and then said DNAs were transformed into BL21 (DE3) (Novagen, Inc.) after confirming desirable DNA without mutagenesis or frame shift had been cloned using DNA sequencing.

### Example 6

### Expression and Refolding of the Protease having Algicidal Activity

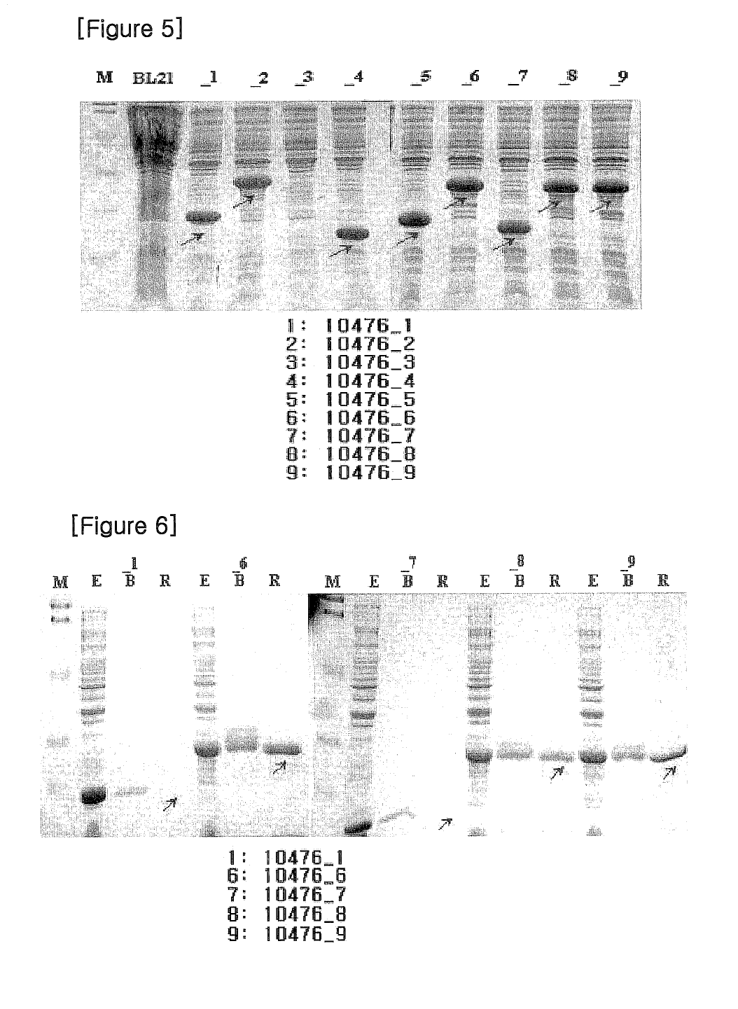

Transformants obtained according to Example 5 were inoculated into LB broth (Difco, Inc.) containing 50 μg/ml kanamycin and incubated at 37° C. for 12 hr. 1% (v/v) of the culture solution was inoculated into the same broth and incubated for 3 hr, and then overexpression of proteases was induced by addition of IPTG (Sigma, Inc.) to the final concentration of 1 mM. To analyze the expression level and time of each protease, SDS-PAGE analysis was performed after sampling at a 1 hr interval. The expression level of proteases at 3 hr after culture is shown in FIG. 5.

As shown in FIG. 5, it was found that all proteases except for the plasmid inserted with PCR product of 10476_{—}3 were expressed.

Also, it was found that most proteases were insoluble proteins by checking where the protease expressed according to the protocol of pET system was produced among soluble, insoluble or periplasmic fraction.

Also, the proteases of 10476_{—}1, 10476_{—}6, 10476_{—}7 (SEQ ID NO: 27), 10476_{—}8 (SEQ ID NO: 26) and 10476_{—}9 obtained above were solubilized by inoculating their insoluble fractions with 8M urea, and then the final proteases were obtained by refolding through substitution with buffer solution (50 mM Tris (pH 8.0), 1 mM CaCl₂) (FIG. 6). As a result, it was found that all proteases were refolded.

### Example 7

### Measurement of the Protease having Algicidal Activity

Protease activity of proteases obtained according to Example 6 was measured using electrophoresis on gelatin substrate gel [Park, H. I., J. Biol. Chem., 275:20540-20544, 2000]. The result is shown in FIG. 7.

As shown in FIG. 7, the proteins of 10476_{—}1, 10476_{—}6, 10476_{—}7 and 10476_{—}8 except for 10476_{—}9 protein showed protease activity.

Also, the band showing activity of 10476_{—}8 protein (SEQ ID NO: 26) containing processing peptide gradually moved down with the passage of time until it showed similar size to the band of 10476_{—}7 protein (SEQ ID NO: 27).

Therefore, it can be expected that processing peptide of 10476_{—}8 protein turns into mature protein by autocleavage with the passage of time.

The result of 10476_{—}1 protein indicates that the presence of N-terminal His-tag inhibits protease activity, and the result of 10476_{—}9 protein indicates that a C-terminal deletion mutant shows no protease activity because of loss of processing.

### Example 8

### Observation of Protein Processing of the Protease having Algicidal Activity

To observe protein processing of the protease having algicidal activity according to the present invention, analysis of protease activity was performed as the same method of Example 7 except 10476_{—}8 protein (SEQ ID NO: 26) which was reacted at 4° C. for a week, and then that was confirmed by SDS-PAGE (FIG. 8).

As shown in FIG. 8, it was found that 10476_{—}8 protein turned into mature protease by degradation of processing peptide 3 days after reaction, and 2 main bands were showed 7 days after reaction. Also, it was founded that autocleavage occured during maturing process, considering the amount of main proteins and low molecular peptides increased with the passage of time.

Also, as a result of analysis of 10476_{—}9 protein as the same method above, 10476_{—}9 protein, a mutant that C-terminal 5 amino acids were deleted containing processing peptide was purified well, but decrease of band size like 10476_{—}8 protein was not observed. Therefore, we might conclude that C-terminal amino acids play an important role because lack of protein processing of 10476_{—}9 protein was the reason that the protein didn't show protease activity.

### Test Example 1

### Identification of Biochemical Characters of the Protease having Algicidal Activity

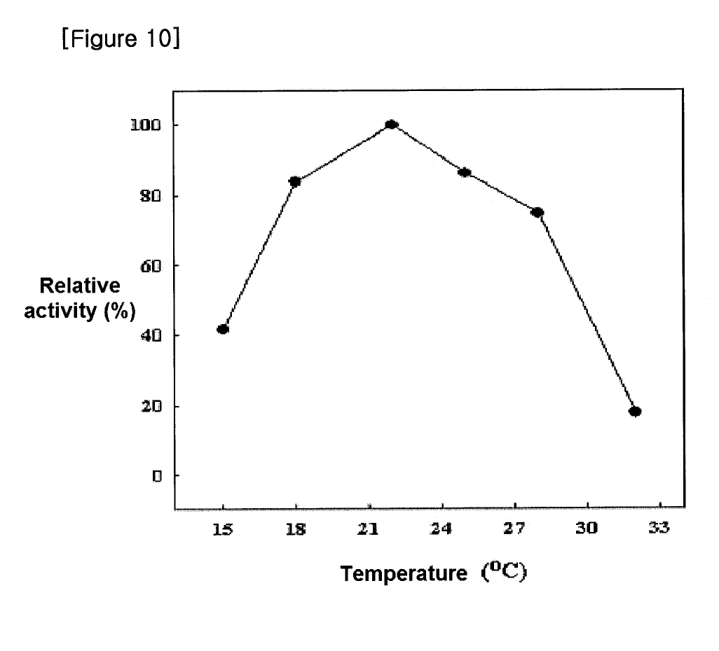

10476_{—}7 protein (SEQ ID NO: 27) containing only mature protein was suspended with SDS in combination with stabilization by using EDTA and resistance to detergent, and refolded by dilution using buffer solution containing 50 mM Tris-HCl, 1 mM EDTA and 1% triton X-100 except SDS, and then protease activity was measured at various temperature and pH according to the method as described above [Park, H. I., J. Biol. Chem., 275:20540-20544, 2000]. The results are shown in FIG. 9 and FIG. 10, respectively.

As shown in FIG. 9 and FIG. 10, 10476_{—}7 protein showed optimal activity at pH 7-8 and about 20° C.

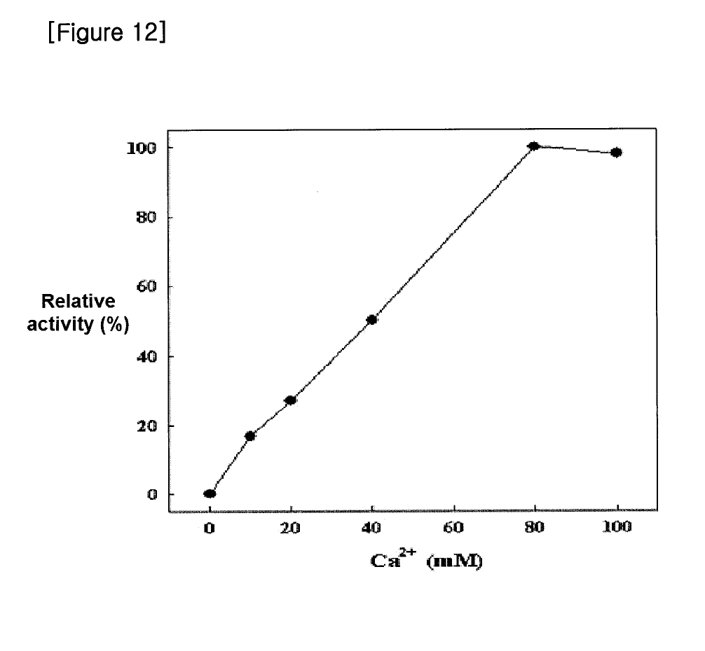

Also, 5 μg of refolded protein was added to buffer solution containing 0.1% gelatin, 50 mM Tris-HCl (pH 8.0), 100 mM KCl and 1 mM zinc or calcium ion, incubated at 20° C. for 2 hr, and then 5% TCA solution (w/v) was added to the solution, followed by stand for 10 min on ice. After centrifugation for 5 min at 10,000 rpm, the protein was measured using the supernatant. Inhibition of protease activity by metal ions was measured by comparing with control group without metal ion. The results are shown in FIG. 11 and FIG. 12.

As shown in FIG. 11, inhibition of 10476_{—}7 protein activity by zinc ion was confirmed.

Also, as shown in FIG. 12, calcium ion was essential for protease activity and maximal activity of protease was observed at 80-100 mM calcium ion.

### Test Example 2

### Analysis of Algicidal Activity of the Protease having Algicidal Activity

To measure algicidal activity of 10476_{—}7 protein (SEQ ID NO:27), 50 μl of said protein (100 μg/ml) was added to the culture solutions of Cochlodinium polykrikoides, Thalassiosira sp., Heterosigma akashiwo, Skeletonema costatum, Alexandrium sp., Chaetoceros cuvisetus and Gymnodniium sp. which were prepared in Example 2. After 24 hr, said culture solutions were stained with 1% Lugol's iodine solution, and then the cell number was counted. Experimental groups with protease (experimental group 1 and 2) were compared with control group without protease in cell number and the degree of inhibition. The results are shown in Table 3.

TABLE 3
Algicidal activity against Cochlodinium polykrikoides

| | Control | experimental | experimental |
|---|---|---|---|
| | group | group 1 | group 2 |
| Cell number | 1,360(130) | 960(30) | 1040(90) |
| (cell/ml) | | | |
| (cell/ml) | 1,330(120) | 940(60) | 900(110) |
| The degree of | — | 40.7 | 30.0 |
| inhibition (%) | | | |

The degree of inhibition (%) = ((Control − experimental)/experimental) × 100
( ): standard deviation

As shown in Table 3, it was confirmed that cell growth was inhibited effectively when Cochladinium polykrikoides was treated with the protease of SEQ ID NO: 1. Also, the protease showed algicidal activity against Thalassiosira sp., Heterosigma akashiwo, Skeletonema costatum, Alexandrium sp., Chaetoceros cuvisetus and Gymnodinium sp.

### Example 9

### Homology Search for a Gene

Base on amino acids sequence of the protease (SEQ ID NO: 1) according to the present invention, which was confirmed algicidal effect, said amino acids sequence was compared with those of marine microorganism using NCBI Blast. As a result, said protease exhibits more than 80% gene coverage and more than 40% identity with a protein from Flavobacterium johnsoniae UW101 (ZP_{—}01247095, SEQ ID NO: 4), Croceibacter atlanticus HTCC2559 (ZP_{—}00951344, SEQ ID NO: 5) and Leeuwenhoekiella blandensis MED217 (ZP_{—}01059780, SEQ ID NO: 6), as shown in FIG. 13. Especially, the part of mature protease which is downstream of processing peptide shows high homology more than 50% to said proteins. As shown in FIG. 14, the protease also shows high homology to Kordia algicida KAOT1_{—}11562 (SEQ ID NO: 7).

### Example 10

### Expression of Proteins with Homology to the Protease having Algicidal Activity

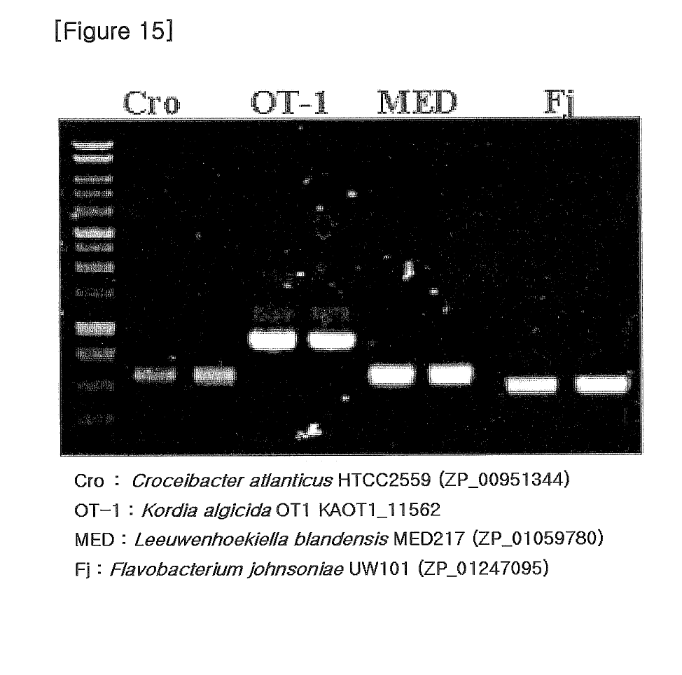

To express proteins with homology to the protease of SEQ ID NO: having algicidal activity, PCR was performed using primer combinations as shown in Table 4 and the gene encoding the protein having amino acids sequence of SEQ ID NO: 4-7, respectively, as a template (SEQ ID NO: 8-11, respectively). Also, PCR was performed as the same method in Example 5 except as noted above. The results are shown in FIG. 15.

TABLE 4

| PCR products | primer | SEQ ID NO. |
|---|---|---|
| ZP_00951344 | HTCC2559m_t | 18 |
| | HTCC2559m_bc | 19 |
| 11562 | OT11562m_t | 20 |
| | OT11562m_bc | 21 |
| ZP_01059780 | MED217m_t | 22 |
| | MED217m_bc | 23 |
| ZP_01247095 | fjohnm_t | 24 |
| | fjohnm_bc | 25 |

And then, each PCR product was cloned into pET24 expression vector as the same method in Example 5, and each protein was overexpressed and analyzed by SDS-PAGE as the same method in Example 6. As a result, all 4 proteins were expressed in high yield but detected in the insoluble fraction. Accordingly, to measure protease activity, each protein was resuspended as the same method in Example 1 (before), and then refolded by dilution in buffer solution without SDS and containing 50 mM Tris-HCl, 1 mM EDTA and 1% Triton X-100. The results are shown in FIG. 16.

As shown in FIG. 16, in case of all proteins except for Croceibacter atlanticus HTCC2559 (SEQ ID NO: 5), the high yield of mature protein was obtained.

### Example 11

### Analysis of Algicidal Activity of Algicidal Proteins having Homology

To check algicidal activity of proteins isolated Flavobacterium johnsoniae UW101 (ZP_{—}01247095, SEQ ID NO: 4), Croceibacter atlanticus HTCC2559 (ZP_{—}00951344, SEQ ID NO: 5), Leeuwenhoekiella blandensis MED217 (ZP_{—}01059780, SEQ ID NO: 6) and Kordia algicida KAOT1_{—}11562 (SEQ ID NO: 7), 50 μl of said protein (100 μg/ml) was added to the culture solutions of Cochlodinium polykrikoides, Thalassiosira sp., Heterosigma akashiwo, Skeletonema costatum, Alexandrium sp., Chaetoceros cuvisetus, and Gymnodium sp., which were prepared in Example 2. After 24 hr, said culture solutions were stained with 1% Lugol's iodine solution, and then the cell number was counted.

Experimental groups with protease (experimental group 1 and 2) were compared with control group without protease in cell number and the degree of inhibition. As a result, the protease showed algicidal activity against Thalassiosira sp., Heterosigma akashiwo, Skeletonema costatum, Alexandrium sp., Chaetoceros cuvisetus and Gymnodinium sp.

TABLE 5
Algicidal activity of proteins inhibiting
growth of red tide microorganism

| | FJ | CRO | MED | | |
|---|---|---|---|---|---|
| | (ZP_01247095) | (ZP_00951344) | (ZP_01059780) | KAOT1_10476 | KAOT1_11562 |
| | (SEQ ID NO: | (SEQ ID NO: | (SEQ ID NO: | (SEQ ID NO: | (SEQ ID NO: |
| | 4) | 5) | 6) | 1) | 7) |
| Cochlodinium | ++++ | ++++ | ++++ | ++++ | ++++ |
| polykrikoides | | | | | |
| Thalassiosire | ++++ | ++++ | ++++ | ++++ | ++++ |
| sp. | | | | | |
| Hererosigma | ++++ | ++++ | ++++ | ++++ | ++++ |
| akashiwo | | | | | |
| Skeletonema | ++ | +++ | ++++ | + | + |
| costatum | | | | | |
| Alexandrium | ++++ | ++++ | ++++ | ++++ | ++++ |
| sp. | | | | | |
| Chaetoceros | ++++ | ++++ | ++++ | ++++ | ++++ |
| cuvisetus | | | | | |
| Gymnodinium | ++++ | ++++ | ++++ | ++++ | ++++ |
| sp. | | | | | |

The degree of inhibition (%) = ((Control − experimental) / experimental) × 100
+ means <25%
++ means 25-50%
++ means 50-75%
++++ means >75% the degree of inhibition

### INDUSTRIAL APPLICABILITY

As described above, the protein having algicidal activity according to the present invention inhibits effectively the growth of red tide microorganism. Therefore, the protein can be used for controlling red tide occurred in marine.

1. A protease having algicidal activity comprising any one amino acids sequence selected from the group consisting of SEQ ID NO: 1, SEQ ID NO: 4 to SEQ ID NO: 7, SEQ ID NO: 26 and SEQ ID NO: 27.

2. The protease having algicidal activity according to claim 1, which is amino acids sequence of SEQ ID NO: 26.

3. The protease having algicidal activity according to claim 1, which is amino acids sequence of SEQ ID NO: 27.

4. The polynucleotide encoding the protease of claim 1.

5. The polynucleotide according to claim 4, which is any one nucleotide sequence selected from the group consisting of SEQ ID NO: 2, SEQ ID NO: 8 to SEQ ID NO: 11, SEQ ID NO: 28 and SEQ ID NO: 29.

6. The polynucleotide according to claim 5, which is nucleotide sequence of SEQ ID NO: 28.

7. The polynucleotide according to claim 5, which is nucleotide sequence of SEQ ID NO: 29.

8. Algicidal formulation comprising the protease of claim 1 as an active composition.

9. A method for controlling red tide comprising the step of introducing the algicidal formulation of claim 8 to the marine region where red tide occurred.

10. A method for controlling red tide comprising the steps of: (a) producing one or more proteases selected from the group consisting of SEQ ID NO: 1, SEQ ID NO: 7 and SEQ ID NO: 27 using Kordia algicida OT-1 KCTC 11320BP and (b) introducing the effective amount of algicidal formulation comprising said protease produced by above step.

11. The method according to claim 10, wherein said algicidal formulation is introduced with Kordia algicida OT1 KCTC 11320BP simultaneously.


SEQUENCE LISTING (29 sequences)

SEQ ID NO: 1 - protein, 278 aa, Kordia algicida
  misc_feature: KAOT1_10476 of Kordia algicida OT-1
MKKIKNLTFALALGLGITMVSCSKDTAVVDEEQDTAIGIPQDVLQKAQSLHFNTFDMQEASFEKPNGKIEEGYMMEGDIFFTRDQLMNMELGGDITSKQYRTNNLVSPGVITIIGYTGNNSNGLTTKMQTGLRWAVDNYNALNLSISFQLTFGTDYQNKDMVVYQVQGGAGGSAGFPSGGNPYKWVKINSGMAPYSNNVHEHVIGHEIGHSIGFRHSDYFSRQSCGQNSNEGSAGVGAIHIPGTPTGWDPTSLMNACFSSSEDGEFNGNDITALNFLY

SEQ ID NO: 2 - DNA, 834 bp, Kordia algicida
  misc_feature: KAOT1_10476 of Kordia algicida OT-1
atgaaaaaaattaaaaacttaacctttgcgttggcattagggttagggatcactatggtatcttgtagtaaagacactgccgtagtagacgaggagcaagacacagctatcggaatcccacaagacgtacttcaaaaagcacaatcacttcacttcaacacattcgatatgcaagaagcatctttcgaaaagccaaatggtaaaatcgaagaaggatatatgatggaaggtgatatcttctttacacgtgaccaattaatgaacatggaactcggtggagatattacaagcaaacaatatcgtacaaacaatttagtttctccaggagtaattacaatcattggatatacaggaaataactctaatggattaacaactaaaatgcaaactggacttagatgggcagtagataactataatgcattaaacctaagtattagtttccaattaacatttggtactgactatcaaaataaagatatggtagtatatcaagtacaaggtggagcaggtggatctgcaggtttcccttcaggaggaaatccatacaagtgggttaaaattaactcaggtatggctccttacagtaacaatgtacatgaacatgtaattggacatgaaataggacactcgattggattccgtcattcagactacttcagcagacaaagttgtggacaaaattcaaatgaaggtagtgcaggagttggagcaatccatattccaggaacacctacaggttgggatccaacatctttaatgaatgcttgtttcagttcttcagaagatggagaatttaacggaaacgacattactgctttaaacttcttatac

SEQ ID NO: 3 - protein, 20 aa, Kordia algicida
  misc_feature: N-terminus of KAOT_1 10476
QYVTNNLVSPGVITIIGYTF

SEQ ID NO: 4 - protein, 276 aa, Flavobacterium johnsoniae
  misc_feature: Flavobacterium johnsoniae UW101 (ZP_01247095)
MKKIKSILILSFTALVLLSCNKEDETVSSGQESLKVTPEVLEKLKSLSLNTSDVQVIQNTSLEGAVEDAFLVEGDIIITQAQLNKMDLHGGITTEQYRTTNLVSAPRTIKVVGLSGTGTTALTTNMRNGLQAAINRYNNLGLSINFTLTFSSSTSGANIVVRRQTGSAGGVAGFPSGGNPYNSVTLYSGLDSYSTNVNAHVAAHEIGHCIGLRHTDWFSRQSCGQNSNEGTAGVGAILIPGTPSGYDATSYMRACFGSNETGAFNANDITALNYLY

SEQ ID NO: 5 - protein, 284 aa, Croceibacter atlanticus
  misc_feature: Croceibacter atlanticus HTCC2559 (ZP_00951344)
MVFVSCEKDNDTNNAEVADTNEVTTGSLETLGVNTDLTPTNLDQATLDLIASKHLSPIGAQEELRYLPDGTSEKAIRIEGDIVMTKAELEELEFNGYSNENAQYSTNALVSPQTITIIGYTGGSQALTSSEQTALQWAVANYNRLNLNINFSLTFGTNYQNKDMVVYNNTVNNPSGAGGSAGFPSGGNPHKFVQIYGLSNYNTNVIEHVITHEIGHSVGFRHTDYFSRQSCGQNTNEGTAGVGANHIPGTPTGYDSTSIMLACFSSGEDGEFNSNDITALNYLY

SEQ ID NO: 6 - protein, 270 aa, Leeuwenhoekiella blandensis
  misc_feature: Leeuwenhoekiella blandensis MED217 (ZP_01059780)
MTSCSEDAEQTTLPEAQPTSNLEVSSEIIQQVEDLGMNANYVRWDDFYFPDGSSEPRLFLEEDVVVTPEQLSSMSANVEESKSGKGDSKQYRTSALVSQGRTISIIGYTGGSQALSQKERTALQWAVANYNRLSGVSISFNLTFGTDYQNKDMVVYNNTVNNPSGAGGSAGFPSNGLPYKFVQIYGLANYDTNVVEHVMTHEIGHSVGFRHTDWFSRQSCGQNVYEGGDANHVSGTPTGYDSTSIMLACFSANEDGEFNANDITALNNMY

SEQ ID NO: 7 - protein, 378 aa, Kordia algicida
  misc_feature: Kordia algicida KAOT1_11562
MKRNFKIQMLYVLLTMLLLAGCQNDTEEELVEEATTAVERVDVKWLAPDDHPVVQLLYSRGYERGTIYETDEHFLAPPDLLYSKDINDYDLSDNGSNAEQAYNTGKLVSLNRMRINVFLDNSIGTDLQTQSVNAMNELNGINNCALFFVRVFNANQAQITIRSDFGAESNNVLGRAGFPSNGRPFDTVTLNVDRLDDFGADIRRNTIIHELGHCVGLRHTDWQANRERSAVNIPGTSANDTGSIMWHTINGGTPFTNGDLTAFRALFPRALRIDVVNEIDDYDYSGEIYVLDNVFVDVFTDGSYSTQTTLNRNVNVSYRINVQEYNHTSGTYYYSRNRTLTAGNNRYYIDDEEEECSPYQGETCTRQDLEIRLATSIL

SEQ ID NO: 8 - DNA, 828 bp, Flavobacterium johnsoniae
  misc_feature: Flavobacterium johnsoniae UW101 (ZP_01247095)
atgaaaaaaattaaatcaatcctgattctgtcatttacagcattagtactattgtcttgtaacaaagaagatgagactgtatcatctggtcaagagtctttaaaagtaacaccagaggtgttggaaaaacttaaatcactttctttgaacacctcagatgtgcaggtgatccaaaatactagtttagaaggtgctgttgaagacgcgttccttgtagaaggggatattatcatcacgcaggcacaattaaacaaaatggatcttcacggaggtattacaacagaacaataccgtactactaatttagtatctgctccaagaacgatcaaagttgttggtttatcaggaactggtacaacagccctgactaccaatatgcgtaacggactgcaggctgctataaacagatacaataatttaggattatctataaactttactttgacttttagttcaagtacttcaggcgcaaacattgtagtgcggagacaaaccggatctgccggcggagtagctggtttcccttcaggaggaaatccatataattcagttaccttatattcaggattagattcttattcaacaaatgtaaacgcacacgttgcggcacatgaaataggacattgtattggtctgcgtcatacagactggttcagccgtcaaagctgcgggcagaattcaaatgaaggaactgctggtgtaggagcaattcttattccgggaacaccttctggatatgatgctacttcttacatgagagcgtgtttcggttcaaacgaaaccggtgctttcaatgctaatgatattacagcattgaactatttatat

SEQ ID NO: 9 - DNA, 852 bp, Croceibacter atlanticus
  misc_feature: Croceibacter atlanticus HTCC2559 (ZP_00951344)
atggtattcgtttcttgtgagaaagataatgatacaaacaacgcagaggttgcagacactaatgaagttacaacaggttctcttgaaactttaggtgttaatacagaccttaccccaacaaatttagatcaagctactttagatttaattgcttctaaacatcttagtcctatcggagctcaagaagagttaagatatttaccagacggtacatcagaaaaagctatacgtatagaaggtgatatcgtaatgactaaagctgaacttgaagagttggagtttaatggttattcaaatgaaaatgctcaatatagtactaacgcattagttagtcctcaaacaattacaattattggttatactggtggaagccaggcgttaactagtagcgagcaaactgcattacaatgggcagttgcaaactataatagattaaaccttaacatcaatttctctttaacttttggtacaaactaccaaaacaaagatatggttgtttacaacaatactgtaaataacccaagtggtgcaggaggatctgcaggtttcccaagtggaggaaacccacataaatttgtacagatttatggattaagtaactataacacaaatgtaattgagcacgttattactcacgaaataggacactctgtaggattccgtcacacagattactttagccgtcagagttgtggtcaaaatacaaacgaaggaactgcaggagttggtgctaaccacatcccaggaactccaacaggatatgattctacgtctattatgttagcttgttttagcagtggtgaagatggagagtttaacagtaatgatattactgctttaaactacctttat

SEQ ID NO: 10 - DNA, 810 bp, Leeuwenhoekiella blandensis
  misc_feature: Leeuwenhoekiella blandensis MED217 (ZP_01059780)
atgacctcttgttcagaagatgctgagcaaactacgttgccagaagcacaacctacttccaaccttgaggtttcttcagaaattatccagcaagttgaagatttaggcatgaacgctaattatgtgcgttgggatgatttttattttcctgatggttcttctgaacctcgtctatttttagaagaagatgttgtcgtaactcctgaacaattatcctcaatgtccgctaacgttgaagaatcaaaatcaggaaaaggtgattctaaacaatatcgcacttctgctttggtaagtcagggaagaacgatttctatcatcggttataccggcggtagtcaggcgctaagccaaaaagagcgtaccgccttacaatgggctgttgcaaattacaatcggttaagcggtgtaagcatcagcttcaatttgacatttggtaccgactatcaaaacaaagacatggtagtttacaacaacacggtaaataacccaagtggcgcaggtggtagtgccggtttcccgagtaatggacttccttataaatttgttcagatctatgggctcgctaattacgacaccaatgttgttgaacacgtaatgacgcacgagataggtcattcggttggatttagacacacagattggtttagtagacaaagctgtggacagaacgtttatgaaggtggtgacgcaaatcacgtttcgggaaccccaacaggttacgactcgacttcaattatgctagcttgctttagtgccaatgaagatggcgaatttaatgccaatgatattaccgcattaaacaatatgtac

SEQ ID NO: 11 - DNA, 1134 bp, Kordia algicida
  misc_feature: Kordia algicida KAOT1_11562
atgaaaagaaattttaaaatccaaatgctgtatgttttgttgacaatgctcctgcttgcaggttgtcaaaacgatacggaagaagaattagtagaagaagcaacaactgctgtagaacgtgttgatgtaaaatggcttgcaccagacgatcatcctgtggtacaattattatacagcagaggttatgaacgaggaacaatctatgaaactgatgagcattttttagcgccaccagatttattgtacagtaaagatatcaacgattatgacctatctgacaatggtagcaatgccgaacaagcctacaacacagggaaattagtatctctaaacagaatgcgtatcaatgtgtttttagacaactctattgggacagatttgcaaacacaatccgtaaatgcaatgaatgaacttaacggaatcaacaattgtgctttattctttgtacgtgttttcaatgccaatcaggcacaaattacgatacgaagtgactttggagcagaatccaataatgttttaggtcgtgcaggttttccatctaacggacgtccatttgatacagtgaccctaaacgttgatcgtttggatgacttcggagctgacattcgtagaaacaccattattcatgaacttggtcattgtgtcggattgcgccatacagattggcaagccaacagagaacgtagtgctgtaaacattcctggtacaagtgccaatgatacaggatcaatcatgtggcataccatcaatggaggaacaccttttacaaacggagatttaactgcttttagagccttatttccacgagctttgcgcatagatgttgtcaatgaaattgacgattatgattacagtggtgaaatttatgttttagacaatgtatttgtcgatgtctttaccgatggaagctacagtacgcaaacaacgcttaaccgaaatgtaaacgtaagttaccgtattaacgtacaagaatacaatcacacctcaggaacttattattacagtcgaaatcgaactttaacggctggtaataatcgttattatattgatgatgaggaagaagaatgttcgccataccaaggagaaacctgtacacgacaagaccttgaaatcagattggcaacttcaatacta

SEQ ID NO: 12 - DNA, 40 bp, Artificial Sequence
  Synthetic Construct; OT1m_t
cgacccggcatatgcaatatcgtacaaacaatttagtttc

SEQ ID NO: 13 - DNA, 37 bp, Artificial Sequence
  Synthetic Construct; OT1m_bc
ctccacatctcgagctagtataagaagtttaaagcag

SEQ ID NO: 14 - DNA, 39 bp, Artificial Sequence
  Synthetic Construct; OT1m_bH
ctccacatctcgaggtataagaagtttaaagcagtaatg

SEQ ID NO: 15 - DNA, 41 bp, Artificial Sequence
  Synthetic Construct; OT1delta5_bc
ctccacatctcgagctaagcagtaatgtcgtttccgttaaa

SEQ ID NO: 16 - DNA, 37 bp, Artificial Sequence
  Synthetic Construct; OT1delta26_bc
ctccacatctcgagctaagatgttggatcccaacctg

SEQ ID NO: 17 - DNA, 38 bp, Artificial Sequence
  Synthetic Construct; OT1p_t
cgacccggcatatgagtaaagacactgccgtagtagac

SEQ ID NO: 18 - DNA, 40 bp, Artificial Sequence
  Synthetic Construct; HTCC2559m_t
cgacccggcatatgcaatatagtactaacgcattagttag

SEQ ID NO: 19 - DNA, 37 bp, Artificial Sequence
  Synthetic Construct; HTCC2559m_bc
ctccacatctcgagttaataaaggtagtttaaagcag

SEQ ID NO: 20 - DNA, 39 bp, Artificial Sequence
  Synthetic Construct; OT11562m_t
cgacccggcatatgcaagcctacaacacagggaaattag

SEQ ID NO: 21 - DNA, 37 bp, Artificial Sequence
  Synthetic Construct OT11562m_bc
ctccacatctcgagttatagtattgaagttgccaatc

SEQ ID NO: 22 - DNA, 40 bp, Artificial Sequence
  Synthetic Construct; MED217m_t
cgacccggcatatgcaatatcgcacttctgctttggtaag

SEQ ID NO: 23 - DNA, 37 bp, Artificial Sequence
  Synthetic Construct; MED217m_bc
ctccacatctcgagctagtacatattgtttaatgcgg

SEQ ID NO: 24 - DNA, 40 bp, Artificial Sequence
  Synthetic Construct; fjohnm_t
cgacccggcatatgcaataccgtactactaatttagtatc

SEQ ID NO: 25 - DNA, 37 bp, Artificial Sequence
  Synthetic Construct; fjohnm_bc
ctccacatctcgagttaatataaatagttcaatgctg

SEQ ID NO: 26 - protein, 251 aa, Kordia algicida
  misc_feature: KAOT1_10476 of Kordia algicida OT-1 lacking signal peptide
VVDEEQDTAIGIPQDVLQKAQSLHFNTFDMQEASFEKPNGKIEEGYMMEGDIFFTRDQLMNMELGGDITSKQYRTNNLVSPGVITIIGYTGNNSNGLTTKMQTGLRWAVDNYNALNLSISFQLTFGTDYQNKDMVVYQVQGGAGGSAGFPSGGNPYKWVKINSGMAPYSNNVHEHVIGHEIGHSIGFRHSDYFSRQSCGQNSNEGSAGVGAIHIPGTPTGWDPTSLMNACFSSSEDGEFNGNDITALNFLY

SEQ ID NO: 27 - protein, 180 aa, Kordia algicida
  misc_feature: KAOT1_10476 of Kordia algicida OT-1 lacking signal and processing peptide
QYRTNNLVSPGVITIIGYTGNNSNGLTTKMQTGLRWAVDNYNALNLSISFQLTFGTDYQNKDMVVYQVQGGAGGSAGFPSGGNPYKWVKINSGMAPYSNNVHEHVIGHEIGHSIGFRHSDYFSRQSCGQNSNEGSAGVGAIHIPGTPTGWDPTSLMNACFSSSEDGEFNGNDITALNFLY

SEQ ID NO: 28 - DNA, 753 bp, Kordia algicida
  misc_feature: KAOT1_10476 of Kordia algicida OT-1 lacking signal peptide
gtagtagacgaggagcaagacacagctatcggaatcccacaagacgtacttcaaaaagcacaatcacttcacttcaacacattcgatatgcaagaagcatctttcgaaaagccaaatggtaaaatcgaagaaggatatatgatggaaggtgatatcttctttacacgtgaccaattaatgaacatggaactcggtggagatattacaagcaaacaatatcgtacaaacaatttagtttctccaggagtaattacaatcattggatatacaggaaataactctaatggattaacaactaaaatgcaaactggacttagatgggcagtagataactataatgcattaaacctaagtattagtttccaattaacatttggtactgactatcaaaataaagatatggtagtatatcaagtacaaggtggagcaggtggatctgcaggtttcccttcaggaggaaatccatacaagtgggttaaaattaactcaggtatggctccttacagtaacaatgtacatgaacatgtaattggacatgaaataggacactcgattggattccgtcattcagactacttcagcagacaaagttgtggacaaaattcaaatgaaggtagtgcaggagttggagcaatccatattccaggaacacctacaggttgggatccaacatctttaatgaatgcttgtttcagttcttcagaagatggagaatttaacggaaacgacattactgctttaaacttcttatac

SEQ ID NO: 29 - DNA, 540 bp, Kordia algicida
  misc_feature: KAOT1_10476 of Kordia algicida OT-1 lacking signal and processing peptide
caatatcgtacaaacaatttagtttctccaggagtaattacaatcattggatatacaggaaataactctaatggattaacaactaaaatgcaaactggacttagatgggcagtagataactataatgcattaaacctaagtattagtttccaattaacatttggtactgactatcaaaataaagatatggtagtatatcaagtacaaggtggagcaggtggatctgcaggtttcccttcaggaggaaatccatacaagtgggttaaaattaactcaggtatggctccttacagtaacaatgtacatgaacatgtaattggacatgaaataggacactcgattggattccgtcattcagactacttcagcagacaaagttgtggacaaaattcaaatgaaggtagtgcaggagttggagcaatccatattccaggaacacctacaggttgggatccaacatctttaatgaatgcttgtttcagttcttcagaagatggagaatttaacggaaacgacattactgctttaaacttcttatac